(19) United States
(12) Patent Application Publication
(10) Pub. No.: US 2011/0230660 A1
(43) Pub. Date: Sep. 22, 2011

(54) METHOD FOR PREPARING A SPIROINDOLINE AND A PRECURSOR THEREOF

(75) Inventors: Stacie Calad, King of Prussia, PA (US); Brian Daniel Doan, King of Prussia, PA (US)

(73) Assignee: Method for Preparing a Spiroindoline

(21) Appl. No.: 13/130,766

(22) PCT Filed: Dec. 3, 2009
(86) PCT No.: PCT/US09/66525

§ 371 Date: May 24, 2011

Related U.S. Application Data
(60) Provisional application No. 61/119,743, filed on Dec. 4, 2008.

(51) Int. Cl. C07D 221/00; C07D 211/00

(52) U.S. Cl. 546/16; 546/246

(57) ABSTRACT

The present invention relates to a method comprising the step of contacting a protected N,N-bis(2-X-ethyl)amine with a (2-fluorophenyl)acetonitrile in the presence of a water-soluble phase transfer reagent, concentrated aqueous base, and an immiscible organic solvent, and under such conditions to form a 4-(2-fluorophenyl)-4-piperidinecarbonitrile in at least a 70% yield, wherein X is a leaving group. The 4-(2-fluorophenyl)-4-piperidinecarbonitrile is useful in preparing spiroindolines, which can be used as precursors of compounds that are modulators of CCR2 receptor.

### RELATED APPLICATIONS

This application claims the benefit of U.S. Provisional Application No. 61/119,743 filed 4 Dec. 2008.

### BACKGROUND OF THE INVENTION

The present invention relates to a method of preparing a 1,2-dihydrospiro[indole-3,4′-piperidine] and a 2-fluorophenyl piperidinecarbonitrile precursor thereof. Compounds of the present invention are useful as a precursor to a class of compounds that modulate CCR2 chemokine receptor.

CCR2 is a chemokine receptor that is expressed on a cell surface of monocycles and some other blood leukocytes. CCR2 binds to the monocyte chemotactic protein MCP-1, and other CC chemokines, which are produced at sites of inflammation and infection. Recruitment of monocytes to inflammatory sites by MCP-1/CCR2 interactions has been implicated in driving the pathogenesis of a number of diseases including multiple inflammatory disorders including rheumatoid arthritis, atherosclerosis, multiple sclerosis, bronchiolitis obliterans syndrome, asthma, allergic rhinitis, eczema, atopic dermatitis, kidney disease, alveolitis, nephritis, liver cirrhosis, congestive heart failure, viral meningitis, cerebral infarction, neuropathy, Kawasaki disease, Alzheimer's disease, stroke, acute nerve injury, HIV infection, AIDS, autoimmune diseases, cancer, sepsis, retinosis, inflammatory bowel disease, transplant arteriosclerosis, idiopathic pulmonary fibrosis, psoriasis, HIV-associated dementia, lupus, erthematosis, hepatitis, pancreatitis, Crohn's disease, endometriosis, metabolic syndrome, ocular indications and diabetes.

U.S. application Ser. No. 12/142,899 discloses a class of spiroindolines that are described as being effective as modulators of CCR2 chemokine receptor. One of the precursors in this series of compounds is 1,1-dimethylethyl 4-(4-chloro-2-fluorophenyl)-4-cyano-1-piperidinecarboxylate, which is disclosed as being prepared by contacting (4-chloro-2-fluorophenyl)acetonitrile and N,N-bis(2-chloroethyl)-t-butylcarbamate in the presence of NaH in DMSO at 85° C. The yield of the desired product is reported to be 38%. The cyclization and deprotection of this product is also described in this U.S. Application.

U.S. Patent Publication No. 2005/0054628 (para 0276) discloses a method for preparing 4-(4-bromophenyl)-4-cyano-piperidine-1-carboxylic acid, t-butyl ester in ˜51% yield. This method uses hexadecyltributylphosphonium bromide as a phase transfer reagent and is carried out in a mixture of toluene and water at 110° C. using 10 M NaOH.

Cammack and Reeves, in J. Heterocyclic Chem., 23, 73 (1986) disclose methyltrioctylammonium chloride and hexadecyltributylphosphonium bromide as preferred phase transfer catalysts for a similar reaction, which was carried out at 100° C. The yield of the desired cyanophenylpiperidine using hexadecyltributylphosphonium bromide was reported as 63%.

Given these results, it would be an advance in the art to discover more efficient ways of preparing 4-(2-fluorophenyl)-4-piperidinecarbonitriles.

### SUMMARY OF THE INVENTION

The present invention relates to a method comprising the step of contacting a protected N,N-bis(2-X-ethyl)amine with a (2-fluorophenyl)acetonitrile in the presence of a water-soluble phase transfer reagent, concentrated aqueous base, and an organic solvent that is immiscible with the concentrated aqueous base, and under such conditions to form a protected 4-(2-fluorophenyl)-4-piperidinecarbonitrile in at least a 70% yield, wherein X is a leaving group.

The present invention also relates to a method comprising the steps of:
a) contacting a protected N,N-bis(2-X-ethyl)amine with a (2-fluorophenyl)acetonitrile in the presence of a water-soluble phase transfer reagent, concentrated aqueous base, and an organic solvent that is immiscible with the concentrated aqueous base, and under such conditions to form a protected 4-(2-fluorophenyl)-4-piperidinecarbonitrile; and either
b1) deprotecting then reducing and cyclizing the protected 4-(2-fluorophenyl)-4-piperidinecarbonitrile to form a 1,2-dihydrospiro[indole-3,4′-piperidine]; or
b2) reducing and cyclizing, the deprotecting the protected 4-(2-fluorophenyl)-4-piperidinecarbonitrile to form a 1,2-dihydrospiro[indole-3,4′-piperidine].

The 4-(2-fluorophenyl)-4-piperidinecarbonitrile and the 1,2-dihydrospiro[indole-3,4′-piperidine] are useful as precursors to a class of spiroindolines, which have been demonstrated to be modulators of CCR2 chemokine receptor.

### DETAILED DESCRIPTION OF THE INVENTION

In a first aspect, the present invention relates to a method comprising the step of contacting a protected N,N-bis(2-X-ethyl)amine with a (2-fluorophenyl)acetonitrile in the presence of a water-soluble phase transfer reagent, concentrated aqueous base, and an organic solvent that is immiscible with the concentrated aqueous base, and under such conditions to form a protected 4-(2-fluorophenyl)-4-piperidinecarbonitrile in at least a 70% yield, wherein X is a leaving group.

The term “a protected N,N-bis(2-X-ethyl)amine” refers to the following structure:

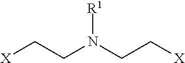

where each X is a leaving group and each R¹ is a protecting group.

Preferably, each X is independently Cl, Br, I, tosylate, mesylate, brosylate, or besylate, with Cl being preferred; and R¹ is a t-butoxycarbonyl (Boc) group or a benzyl-O—C(O)—(CBz) group, with t-butoxycarbonyl being preferred. Accordingly, the preferred protected N,N-bis(2-X-ethyl)amine is 1,1-dimethylethyl bis(2-chloroethyl)carbamate:

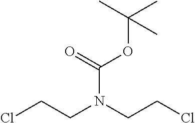

### 1,1-dimethylethyl bis(2-chloroethyl)carbamate

The term “a (2-fluorophenyl)acetonitrile” refers to the following structure:

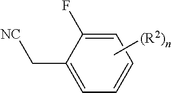

where each R² is independently halo, CF₃, C₁-C₄-alkyl, C₁-C₄-alkoxy, OCF₃, CN, C₁-C₆-alkyl-C(O)—NH—, C₁-C₆-alkyl-NH—C(O)—, —CH₂—N(R³)₂, —CH₂—O—R⁴, C₁-C₄—S(O)ᵣ—, COOH, or heteroaryl; wherein
each R³ is independently H, C₁-C₄-alkyl, or, together with the nitrogen atom to which they are attached, form a 5- or 6-membered heterocycloalkyl group;
R⁴ is H, C₁-C₆-alkyl, benzyl, or phenyl;
r is 0, 1, or 2; and
n is 0, 1, or 2; preferably n is 1.

Preferably, each R² is independently Cl, F, Br, CF₃, CN, CH₃, OCF₃, C₁-C₄—S(O)ᵣ—, or methoxy; more preferably, each R² is independently CH₃, F, Cl, or CN; most preferably, R² is Cl.

The (2-fluorophenyl)acetonitrile is preferably (4-chloro-2-fluorophenyl)acetonitrile.

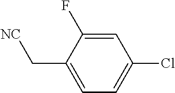

### (4-chloro-2-fluorophenyl)acetonitrile

The term “a protected 4-(2-fluorophenyl)-4-piperidinecarbonitrile” is represented by the following structure:

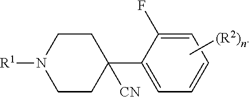

where R¹, R², and n are as previously defined. A particularly preferred 4-(2-fluorophenyl)-4-piperidinecarbonitrile is 1,1-dimethylethyl 4-(4-chloro-2-fluorophenyl)-4-cyano-1-piperidinecarboxylate:

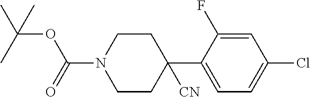

### 1,1-dimethylethyl 4-(4-chloro-2-fluorophenyl)-4-cyano-1-piperidinecarboxylate

The protected 4-(2-fluorophenyl)-4-piperidinecarbonitrile is advantageously deprotected, preferably in situ, by contacting this compound with a suitable agent that removes the protecting group. For example, a Boc-protected 4-(2-fluorophenyl)-4-piperidinecarbonitrile can be converted to the corresponding 4-(2-fluorophenyl)-4-piperidinecarbonitrile by addition of HCl in a suitable solvent such as dioxane. The deprotected product is preferably isolated as its acid salt, as illustrated.

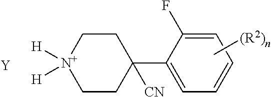

where Y is a counterion such as Cl⁻, Br⁻, SO₄²⁻, or HSO₄⁻.

The deprotected product can be cyclized to the corresponding spiroindoline by reduction followed by cyclization using a suitable reducing agent such as modified lithium aluminum hydride in accordance with the following scheme:

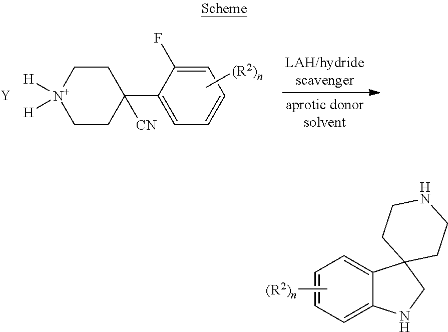

The term “modified lithium aluminum hydride” refers to LAH that has been treated with a hydride scavenger followed by contact of the modified reagent with the deprotected product in the presence of a aprotic donor solvent, including THF, 2-methyl-THF, glyme, t-butylmethylether, diglyme, diethyl ether, and dioxane. As used herein, the term “hydride scavenger” is a reagent that consumes a single hydride from the LAH. Examples of suitable hydride scavengers include protic and reactive carbonyl compounds, including ethanol, methanol, isopropanol, acetone, and ethyl acetate.

The term “water-soluble phase transfer catalyst” refers to a phase transfer catalyst that is preferentially partitioned into the aqueous phase of a biphasic system that contains an aqueous base and an immiscible organic solvent. Preferably, the phase transfer reagent is completely water soluble at the concentrations used and therefore virtually completely partitioned into the aqueous base. Preferably, the water-soluble phase transfer reagent is a water-soluble tetraalkylammonium salt phase transfer reagent such as methyl tributyl ammonium chloride, (commercially available as Aliquat® 175 quaternary ammonium salt) or tetrabutyl ammonium bromide (commercially available as Aliquat® 100 quaternary ammonium salt). Methyl tributyl ammonium chloride is a more preferred water-soluble phase transfer reagent.

As used herein “immiscible organic solvent” refers to one or more organic solvents that form a separate and distinct phase with the aqueous basic phase. Examples of such solvents include toluene, THF, dichloromethane, chloroform, hexanes, cyclohexane, heptane, isopropyl acetate, and methyl t-butyl ether, as well as combinations thereof.

The concentrated aqueous base is preferably a 10% to about a 50% w/w aqueous hydroxide such as LiOH, NaOH, or KOH. Aqueous NaOH is a preferred base, with 50% w/w aqueous NaOH being more preferred. The reaction is also typically carried out at a temperature in the range of about 25° C., more preferably from about 35° C., to about 60° C., more preferably to about 50° C.

### EXPERIMENTAL

The following example is for illustrative purposes only and is not intended to limit the scope of the invention.

### Example 1

### 1,1-Dimethylethyl 4-(4-chloro-2-fluorophenyl)-4-cyano-1-piperidinecarboxylate

To a stirred solution of (4-Chloro-2-fluorophenyl)acetonitrile (8.00 g, 47.2 mmol) and N,N-bis(2-chloroethyl)-t-butylcarbamate (11.42 g, 47.2 mmol) in toluene (50 mL) was added aqueous 50% NaOH w/w (40 mL) and Aliquat® 175 methyl tributyl ammonium chloride (75% w/w in water, 1.55 mL, 4.72 mmol). The reaction mixture was heated to 40° C. and stirred vigorously (700 rpm). After 14 h, the reaction mixture was cooled to 23° C. and diluted with toluene (20 mL) and water (100 mL). The layers were separated and the aqueous layer was extracted with toluene (40 mL). The combined organic layers were washed with 5% HCl (40 mL) and saturated aqueous NaOH (40 mL). The organic layer was dried over anhydrous sodium sulfate and filtered. Analysis of the organic filtrates showed 1,1-dimethylethyl 4-(4-chloro-2-fluorophenyl)-4-cyano-1-piperidinecarboxylate (15.6 g by w/w assay, quantitative).

### Comparative Example

A preparation of 1,1-dimethylethyl 4-(4-chloro-2-fluorophenyl)-4-cyano-1-piperidinecarboxylate that is not within the scope of the process of the present invention is disclosed in U.S. application Ser. No. 12/142,899 as Intermediate 6 is reproduced in the following Comparative Example.

(4-Chloro-2-fluorophenyl)acetonitrile (2.3 g, 13.7 mmol, 1.0 equiv) was added to a suspension of NaH (2.1 g, 52.5 mmol, 3.8 equiv) in DMSO (20 mL) at 23° C. The resulting yellow suspension was stirred for 10 min and the color turned red-brown. Boc-N(CH₂CH₂Cl) (N,N-bis(2-chloroethyl)-t-butylcarbamate) (3.7 g, 15.3 mmol, 1.1 equiv) in DMSO (20 mL) was added to the reaction mixture (bubbling observed) and the resulting suspension was heated to 85° C. with stirring for an additional 1.5 h. The reaction mixture was cooled to 23° C. then poured onto a 1:1 mixture of ethyl acetate and hexanes (300 mL). The organic fraction was washed with water (100 mL) and a saturated aqueous solution of NaCl (100 mL). The organic layer was dried over anhydrous sodium sulfate. The dried solution was then filtered and the filtrate was concentrated. The residue was purified by flash silica chromatography (0%-30% ethyl acetate in hexanes) to afford the bis-alkylation product 1,1-dimethylethyl 4-(4-chloro-2-fluorophenyl)-4-cyano-1-piperidinecarboxylate (1.9 g, 5.6 mmol, 38%) as a yellow crystalline solid. MS (ES) m/e 239 [M−Boc+H]⁺.

Surprisingly, it has been discovered that the use of a phase transfer reagent such as methyl tributyl ammonium chloride dramatically increased the yield of the desired intermediate, from about 38% to, on average, 73%, with the best yield being quantitative.

What is claimed is:

1. A method comprising the step of contacting a protected N,N-bis(2-X-ethyl)amine with a (2-fluorophenyl)acetonitrile in the presence of a water-soluble phase transfer reagent, concentrated aqueous base, and an immiscible organic solvent, and under such conditions to form a protected 4-(2-fluorophenyl)-4-piperidinecarbonitrile in at least a 70% yield, wherein X is a leaving group.

2. The method of claim 1 wherein the protected N,N-bis(2-X-ethyl)amine is represented by the following structure: 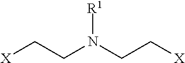
wherein each X is independently Cl, Br, I, tosylate, mesylate, brosylate, or besylate; and R¹ is a t-butoxycarbonyl group or a benzyl-O—C(O)— group;
the (2-fluorophenyl)acetonitrile is represented by the following structure: 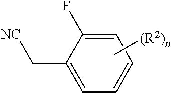
where each R² is independently Cl, F, Br, CF₃, CN, CH₃, OCF₃, C₁-C₄—S(O)ᵣ—, or methoxy; and
the 4-(2-fluorophenyl)-4-piperidinecarbonitrile is represented by the following structure: 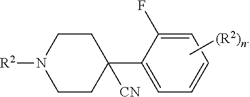
or a salt thereof.

3. The method of claim 2 wherein the water-soluble phase transfer reagent is methyl tributyl ammonium chloride or tetrabutyl ammonium bromide.

4. The method of claim 2 wherein the water-soluble phase transfer reagent is methyl tributyl ammonium chloride.

5. The method of claim 2 wherein each X is Cl and R¹ is a t-butoxycarbonyl group or a benzyloxycarbonyl group.

6. The method of claim 2 wherein R¹ is a t-butoxycarbonyl group.

7. The method of claim 2 wherein the (2-fluorophenyl)acetonitrile is (4-chloro-2-fluorophenyl)acetonitrile, and the protected N,N-bis(2-X-ethyl)amine is 1,1-dimethylethyl bis(2-chloroethyl)carbamate.

8. The method of claim 2 wherein the concentrated aqueous base is about 50% w/w aqueous NaOH and reaction is carried out at a temperature in the range of from 25° C. to about 60° C.

9. The method of claim 2 wherein the reaction is carried out at temperature in the range of from 35° C. to about 50° C.

10. The method of claim 2 wherein the protected 4-(2-fluorophenyl)-4-piperidinecarbonitrile is deprotected to form either the 4-(2-fluorophenyl)-4-piperidinecarbonitrile or an acid salt of the 4-(2-fluorophenyl)-4-piperidinecarbonitrile.

11. The method of claim 10 wherein either the 4-(2-fluorophenyl)-4-piperidinecarbonitrile or the acid salt of the 4-(2-fluorophenyl)-4-piperidinecarbonitrile is converted to a 1,2-dihydrospiro[indole-3,4′-piperidine] by neutralization, reduction, and cyclization of the acid salt of the 4-(2-fluorophenyl)-4-piperidinecarbonitrile.

12. The method of claim 11 wherein either the 4-(2-fluorophenyl)-4-piperidinecarbonitrile or the acid salt of the 4-(2-fluorophenyl)-4-piperidinecarbonitrile is converted by treatment with lithium aluminum hydride in a suitable solvent.

13. A method comprising contacting (4-chloro-2-fluorophenyl)acetonitrile with 1,1-dimethylethyl bis(2-chloroethyl)carbamate in the presence of:
a) a concentrated aqueous base;
b) an immiscible organic solvent; and
c) a water-soluble phase transfer reagent;
at a temperature in the range of from about 25° C. to about 60° C. to form 1,1-dimethylethyl 4-(4-chloro-2-fluorophenyl)-4-cyano-1-piperidinecarboxylate.

14. The method of claim 13 wherein the concentrated aqueous base is 50% w/w NaOH.

15. The method of claim 13 wherein the water-soluble phase transfer reagent is methyl tributyl ammonium chloride and the immiscible organic solvent is toluene.

16. The method of claim 13 wherein the 1,1-dimethylethyl 4-(4-chloro-2-fluorophenyl)-4-cyano-1-piperidinecarboxylate is contacted with HCl to form the HCl salt of 4-(4-chloro-2-fluorophenyl)-4-piperidinecarbonitrile.

17. The method of claim 16 wherein the HCl salt of 4-(4-chloro-2-fluorophenyl)-4-piperidinecarbonitrile is contacted with lithium aluminum hydride under such conditions to form 6-chloro-1,2-dihydrospiro[indole-3,4′-piperidine].

18. A method comprising the steps of:
a) contacting a protected N,N-bis(2-X-ethyl)amine with a (2-fluorophenyl)acetonitrile in the presence of a water-soluble phase transfer reagent, concentrated aqueous base, and the immiscible organic solvent, and under such conditions to form a protected 4-(2-fluorophenyl)-4-piperidinecarbonitrile; and either
b1) deprotecting then reducing and cyclizing the protected 4-(2-fluorophenyl)-4-piperidinecarbonitrile to form a 1,2-dihydrospiro[indole-3,4′-piperidine]; or
b2) reducing and cyclizing, the deprotecting the protected 4-(2-fluorophenyl)-4-piperidinecarbonitrile to form a 1,2-dihydrospiro[indole-3,4′-piperidine].

19. The method of claim 18 wherein:
the protected N,N-bis(2-X-ethyl)amine is 1,1-dimethylethyl bis(2-chloroethyl)carbamate;
the (2-fluorophenyl)acetonitrile is (4-chloro-2-fluorophenyl)acetonitrile;
the water-soluble phase transfer reagent is methyl tributyl ammonium chloride; and the protected 4-(2-fluorophenyl)-4-piperidinecarbonitrile is 1,1-dimethylethyl 4-(4-chloro-2-fluorophenyl)-4-cyano-1-piperidinecarboxylate, which is deprotected with HCl to form the HCl salt of 1,1-dimethylethyl 4-(4-chloro-2-fluorophenyl)-4-cyano-1-piperidinecarboxylate, and reduced and cyclized with modified lithium aluminum hydride in the presence of an aprotic donor solvent to form 6-chloro-1,2-dihydrospiro[indole-3,4′-piperidine].
